(19) United States
(12) Patent Application Publication
(10) Pub. No.: US 2010/0219335 A1
(43) Pub. Date: Sep. 2, 2010

(54) Liquid Chromatography-Mass Spectrometry Analysis of Samples Using Ionic Eluent Comprising a Volatile Ionic Salt

(76) Inventors: Angela Bardotti, Siena (IT); Alessandro Pianigiani, Siena (IT)

Correspondence Address: NOVARTIS VACCINES AND DIAGNOSTICS INC., INTELLECTUAL PROPERTY- X100B, P.O. BOX 8097, Emeryville, CA, 94662-8097, US

(21) Appl. No.: 12/085,507

(22) PCT Filed: Dec. 5, 2006
(86) PCT No.: PCT/IB2006/003918

§ 371 Date: Mar. 2, 2010

(30) Foreign Application Priority Data
Dec. 5, 2005 (GB) 0524782.0

(51) Int. Cl. H01J 49/04; H01J 49/26; A61K 39/02

(52) U.S. Cl. 250/282; 250/288; 424/234.1

(57) ABSTRACT

The use of an ionic eluent in ion exchange chromatography for analysing a sample comprising an analyte, wherein the ionic eluent comprises a volatile ionic salt, in particular an ammonium salt, is described. Such eluents are shown to be compatible with mass spectrometry (MS), providing clean mass spectra of the analyte. Furthermore, eluates from ion exchange chromatography may be advantageously analysed with MS without additional on-line or off-line devices for desalting or suppressing salts in the eluent. Important information concerning the chemical structure and composition of a sample may therefore be obtained with ion chromatography-MS by utilising the invention. The invention also provides a method of analysing a sample (e.g. a vaccine) comprising an analyte (e.g. a saccharide) by ion chromotography-MS by employing an ionic eluent, wherein the ionic eluent comprises a volatile ionic salt. An apparatus for analysing a sample comprising an analyte is also disclosed.

All documents cited herein are incorporated by reference in their entirety.

### TECHNICAL FIELD

This invention is in the field of analysis of eluates from chromatographic separation. In particular, this invention concerns the analysis and quality control of vaccines that include saccharides (e.g. bacterial capsular saccharides).

### BACKGROUND ART

Immunogens comprising capsular saccharide antigens conjugated to carrier proteins are well known in the art. Conjugation converts T-independent antigens into T-dependent antigens, thereby enhancing memory responses and allowing protective immunity to develop, and the prototype conjugate vaccine was for Haemophilus influenzae type b (Hib) [e.g. see chapter 14 of ref. 1]. Since the Hib vaccine, conjugated saccharide vaccines for protecting against Neisseria meningitidis (meningococcus) and against Streptococcus pneumoniae (pneumococcus) have been developed. Other organisms where conjugate vaccines are of interest are Streptococcus agalactiae (group B streptococcus) [2], Pseudomonas aeruginosa [3] and Staphylococcus aureus [4].

Conjugate vaccines for N. meningitidis serogroup C have been approved for human use, and include Menjugate™ [5], Meningitec™ and NeisVac-C™. Mixtures of conjugates from each of serogroups A, C, W135 and Y have been reported [e.g. refs. 6-9], including the Menactra™ product. Other mixtures of conjugated antigens include: (i) meningococcal A/C mixtures [10,11]; (ii) the PrevNar™ product [12] containing seven pneumococcal conjugates; (iii) mixed meningococcal and Hib conjugates [13,14]; and (iv) combined meningococcal, pneumococcal and Hib conjugates [15].

Where saccharides are included in vaccines and other biological products then regulatory authorities generally require their characterisation. A common technique used for saccharide characterisation is anion chromatography, and in particular high performance anion exchange chromatography (HPAEC), followed by saccharide detection, e.g. pulsed amperometric detection (PAD) [16,17].

Mass spectrometry (MS) is a well-known analytical technique. The use of a mass spectrometer on-line with a chromatographic separation system has been developed as an important technique for the analysis of analytes, in particular in the identification of target and unknown compounds in samples. Reversed-phase liquid chromatography (i.e. with a non-polar stationary phase) is typically employed, thus allowing the selection of mobile phases that do not significantly influence the performance of the mass spectrometer.

Difficulties exist, however, when combining mass spectrometry with ion exchange chromatography, especially anion exchange chromatography (e.g. HPAEC). Anion exchange chromatography involves eluting the analyte with an ionic eluent, typically phosphate, sodium (e.g. sodium hydroxide and/or sodium acetate) or phosphoric acid buffers. When coupled with MS, these eluents cause excessive baseline noise and spiking in the mass spectrum, thus significantly degrading the analytical data. To date this problem has been addressed by including either off-line desalting of the chromatographic eluent prior to MS analysis, or by employing an on-line ion suppression system. Both approaches increase the cost and complexity of an ion chromatography-MS approach.

It is an object of the invention to provide further and improved methods and systems for performing ion chromatography-MS characterisation of analytes, e.g. saccharides. In particular, it is an object to overcome the difficulties in the MS analysis of ion chromatography eluates that arise with known ionic eluents.

### DISCLOSURE OF THE INVENTION

The inventors have discovered that these difficulties can be overcome by employing an ionic eluent comprising a volatile ionic salt, in particular an ammonium salt, in the ion exchange chromatography. In particular, it has been discovered that such eluents are compatible with MS, providing clean mass spectra of the analyte. Consequently, eluates from ion exchange chromatography may be advantageously analysed with MS without additional on-line or off-line devices for desalting or suppressing salts in the eluent. Important information concerning the chemical structure and composition of a sample may therefore be obtained, with ion chromatography-MS by utilising the invention.

Therefore, the invention can advantageously allow on-line, high throughput analysis of analytes, particularly saccharides. The invention can provide also benefits in increased speed, reduced cost of analysis and increased sensitivity, accuracy and reproducibility.

According to a first aspect of the invention, an ionic eluent comprising a volatile ionic salt is used in ion chromatography-MS analysis. Thus, the invention provides a method of analysing a sample (e.g. a vaccine) comprising an analyte (e.g. a saccharide) comprising the steps of: (i) eluting the analyte from an ion exchange chromatography column with an ionic eluent to provide an eluate comprising the analyte, wherein the ionic eluent comprises a volatile ionic salt and (ii) analysing the eluate by MS. The invention also provides an apparatus for analysing a sample comprising an analyte, comprising: (i) a reservoir containing an ionic eluent comprising a volatile ionic salt, (ii) an ion exchange chromatography column for eluting the analyte, wherein the column is arranged to receive eluent from the reservoir, and (iii) a mass spectrometer arranged to receive eluate from the column. The invention further provides the use of an ionic eluent in ion exchange chromatography for analysing a sample comprising an analyte, wherein the ionic eluent comprises a volatile ionic salt.

According to a second aspect of the invention, there is provided a method of eluting an analyte from an ion exchange chromatography column, wherein the analyte is eluted using an ionic eluent comprising a volatile ionic salt. The invention further provides the eluate obtained by this chromatographic method of the invention (e.g. comprising a saccharide and a volatile ionic salt). The invention also provides the use of an ionic eluent for eluting an analyte from an ion exchange chromatography column, wherein the ionic eluent comprises a volatile ionic salt.

According to a third aspect of the invention, there is provided a method of analysing the eluate of the second aspect of the invention by MS. The invention also provides the use of MS for analysing the eluate of the second aspect of the invention.

The invention further provides a bulk pharmaceutical composition comprising as an active ingredient an analyte, wherein a sample of the bulk pharmaceutical composition has been analysed using a method of the invention. The invention also provides a pharmaceutical composition drawn from the bulk pharmaceutical composition. A preferred pharmaceutical composition is an immunogenic composition, such as a vaccine, comprising a bacterial capsular saccharide analyte.

According to a fourth aspect of the invention, there is provided an analyte (e.g. a saccharide) in an ionic buffer, wherein the ionic buffer comprises a volatile ionic salt. When the saccharide is of known length and/or structure, it may be used as a standard, e.g. for calibration of the apparatus of the first aspect of the invention.

### The Volatile Ionic Salt

The volatile ionic salts employed in the invention include ionic salts capable of decomposing, or reacting with another component of the eluate (e.g. hydroxide), to form at least one volatile compound which can evaporate from the eluate before or during ionisation in the mass spectrometer. Preferably, the at least one volatile compound can evaporate at room temperature and atmospheric pressure. Alternatively, the at least one volatile compound can evaporate at an elevated temperature (e.g. from room temperature to 60° C.) or reduced pressure (e.g. 10,000 Pa to atmospheric pressure), provided that the saccharide is not substantially degraded or evaporated from the eluate. Thus, the volatile compound substantially (e.g. >95%, preferably >99%, more preferably >99.9% of the volatile compound) exits the eluate before MS to avoid degrading the mass spectrum. Preferably, the volatile compound substantially exits the eluate within 1 hour, preferably within 10 minutes, more preferably within 5 minutes, still more preferably within 1 minute from eluting the analyte from the ion exchange chromatography column. Furthermore, it is preferred that any remaining volatile ionic salt or volatile compound does not degrade the mass spectrum. Preferably any less volatile and non-volatile compounds formed which remain in the eluent (e.g. H₂O) do not degrade the mass spectrum and are preferably non-ionic.

Preferred volatile ionic salts useful in the invention are ammonium salts, wherein the NH₄⁺ ion may combine with OH⁻ ions present to form NH₃, which is volatile, and H₂O. Examples of ammonium salts useful in the invention include, but are not limited to, ammonium acetate, ammonium benzoate, ammonium bicarbonate, ammonium bromide, ammonium carbamate, ammonium carbonate, ammonium chloride, ammonium formate, ammonium hydrogen phosphate, ammonium hydrogen sulfate, ammonium hydroxide, ammonium nitrate, ammonium oxalate, ammonium phosphate, ammonium sulfate, ammonium tartrate, and mixtures thereof. Particularly preferred ammonium salts include ammonium acetate, ammonium bicarbonate, ammonium carbamate, ammonium carbonate, ammonium formate and ammonium hydroxide, and mixtures thereof. Ammonium hydroxide, ammonium acetate, and mixtures thereof, are especially preferred. The counter-ion to the ammonium ion may (i) remain in the eluate, (ii) react with another component of the eluate to form a product (preferably non-ionic) which remains in the eluate, and/or (iii) react with another component of the eluate to form a product which is itself volatile and evaporates before or during the ionisation of the eluate in the mass spectrometer.

The invention is preferably employed for analysing saccharides, i.e. compounds typically having a molecular weight ≧180 Da. Therefore, the volatile compound preferably has a molecular weight <180 Da, preferably <100 Da, more preferably <50 Da, so that any remaining volatile compound which is detected by MS does not interfere with the mass spectrum in the saccharide region. For similar reasons, it also preferred that the component ions of the volatile ionic salt have a formula weight <180 Da, preferably <100 Da, more preferably <50 Da.

The volatile ionic salt will typically be present at a concentration between 0.0005 to 1 M.

### The Liquid Chromatography (LC) Column

The present invention may be applied to a variety of liquid chromatography columns, but it is preferably used with high performance liquid chromatography (HPLC). Preferred chromatography used in the present invention is ion exchange chromatography, e.g. high performance anion exchange chromatography (HPAEC) or by high performance cation exchange chromatography (HPCEC). Preferred ion exchange chromatography used in the present invention is HPAEC.

Preferred columns are those that spontaneously retain the analyte such that the analyte has to be eluted from the column. Elution from the chromatography column can be an isocratic elution or a gradient elution. For eluting analytes from anion exchange columns then the eluent will generally be basic e.g. the pH will be >8, >9, >10, >11, >12, >13, etc. Hydroxide salts (e.g. NH₄OH) can be used to achieve the desired pH, and hydroxide ions are typical for use in anion exchange eluents.

Preferred HPAEC columns are the hydroxide-selective “IonPac AS” columns marketed by Dionex, such as the AS11 column, with alkanol quaternary ammonium functional groups.

Typically, the methods of the invention will involve an initial step of loading the ion exchange chromatography column with the sample. The sample may be loaded onto an unprepared ion exchange chromatography column, or, more usually, the column may have been pre-prepared by washing and/or equilibrating. After loading, the loaded column may also be washed, to remove contaminants in the sample from the column, and/or re-equilibrated prior to elution. Typically the washing and re-equilibration will be carried out with an ionic solution, e.g. where gradient elution is employed, the solution used at the beginning of the gradient elution. Preferably, the column is washed by elution with a gradient that separates analytes and contaminants, while contaminants more tightly bound to the column are eluted with a final washing.

### The Analyte

The invention is used to analyse the eluate from a liquid chromatography column. The eluate will be the result of chromatographic separation of one or more analytes in a sample.

The invention is particularly useful for analysing saccharide and polypeptide analytes. Saccharide analytes may be polysaccharides (e.g. with a degree of polymerisation of >10, e.g. 20, 30, 40, 50, 60 or more), oligosaccharides (e.g. with a degree of polymerisation of from about 4 to about 10), or monosaccharides. Oligosaccharides and monosaccharides may be the result of depolymerisation and/or hydrolysis of a parent polysaccharide e.g. the analyte may be a saccharide-containing fragment of a larger saccharide.

Preferred saccharide analytes are bacterial saccharides, and particularly bacterial capsular saccharides e.g. from Neisseria meningitidis (serogroups A, B, C, W135 or Y), Streptococcus pneumoniae (serotypes 4, 6B, 9V, 14, 18C, 19F, or 23F), Streptococcus agalactiae (types 1a, 1b, II, III, IV, V, VI, VII, or VIII), Haemophilus influenzae (typeable strains: a, b, c, d, e or f), Pseudomonas aeruginosa, Staphylococcus aureus, Streptococcus mutans, etc. Other saccharide analytes include glucans (e.g. fungal glucans, such as those in Candida albicans), and fungal capsular saccharides e.g. from the capsule of Cryptococcus neoformans.

The N. meningitidis serogroup A capsule is a homopolymer of (α1→6)-linked N-acetyl-D-mannosamine-1-phosphate. The N. meningitidis serogroup B capsule is a homopolymer of (α2→8) linked sialic acids. The N. meningitidis serogroup C capsular saccharide is a homopolymer of (α2→9) linked sialic acid. The N. meningitidis serogroup W135 saccharide is a polymer having sialic acid-galactose disaccharide units [→4)-D-Neup5Ac(7/9OAc)-α-(2→6)-D-Gal-α-(1→]. The N. meningitidis serogroup Y saccharide is similar to the serogroup W135 saccharide, except that the disaccharide repeating unit includes glucose instead of galactose [→4)-D-Neup5Ac(7/9OAc)-α-(2→6)-D-Glc-α-(1→]. The H. influenzae type b capsular saccharide is a polymer of ribose, ribitol, and phosphate [‘PRP’, (poly-3-β-D-ribose-(1, 1)-D-ribitol-5-phosphate)].

In addition to being useful for analysing full-length capsular saccharides, the invention can be used with oligosaccharide fragments of them.

Other preferred saccharide antigens are those cleaved from glycoconjugates e.g. from saccharide-protein conjugate vaccine antigens. Of the three N. meningitidis serogroup C conjugated vaccines that have been approved for human use, Menjugate™ [18] and Meningitec™ are based on oligosaccharides, whereas NeisVac-C™ uses full-length polysaccharide.

Other preferred saccharide antigens are eukaryotic saccharides e.g. fungal saccharides, plant saccharides, human saccharides (e.g. cancer antigens), etc.

Saccharides that are charged (e.g. anionic) at neutral pH are preferred analytes, for example saccharide analytes with multiple phosphate and/or multiple carboxylate groups. The invention is thus particularly useful for analysing polyanionic saccharide analytes.

Other preferred analytes are lipopolysaccharides and lipooligosaccharides, e.g. lipid A of N. meningitidis serogroup B.

The invention is particularly useful for use with analytes that include various saccharides of different lengths e.g. different fragments of the same parent saccharide.

The analyte will generally be in aqueous solution, and this solution will have a high pH and high salt concentration, as a result of HPAEC.

Thus the eluates analysed by the methods of the invention can include these analytes or can be suspected of including them.

Preferred polypeptide analytes are bacterial polypeptides and viral polypeptides.

### The Sample

It is not essential to the invention that the sample contains a particular analyte of interest as the invention may be usefully employed to determine the presence or absence of that particular analyte. Moreover, a step of analysing an analyte which leads to a negative result, i.e. the absence of analyte, is still a step of analysing the sample for the analyte. However, it is preferred that the sample is suspected to contain (and preferably contains) the analyte of interest.

The sample will generally be in aqueous solution.

The invention is particularly useful for analysing an analyte (e.g. a saccharide) in a vaccine. Preferred samples are glycoconjugate vaccines, which may be single or combined (e.g. a combined glycoconjugate vaccine comprising more than one type of glycoconjugate immunogen).

Problems when dealing with conjugate vaccines include stability and batch-to-batch consistency. In Hib vaccines, for instance, catalytic depolymerisation of the saccharide has been reported [19], and conjugates of the serogroup A meningococcus capsule are readily hydrolysed [20]. Instability of conjugates undesirably leads to a reduction in effective dose of immunogenic conjugate over time, variation between batches, and increased levels of uncharacterised breakdown products. Consequently, hydrolysis of the glycoconjugates to free (i.e. unconjugated) saccharide needs to be monitored in the formulated vaccines alone or when in combination with other vaccines. The present invention may be employed to monitor free (i.e. unconjugated) saccharide or conjugated saccharide in a vaccine. Preferably, the invention is used to monitor free saccharide.

### Conjugates

The conjugated saccharides are covalently linked saccharide-carrier conjugates. Covalent conjugation is used to enhance immunogenicity of saccharides by converting them from T-independent antigens to T-dependent antigens, thus allowing priming for immunological memory. Conjugation is particularly useful for paediatric vaccines and is a well known technique [e.g. reviewed in refs. 21 to 30]. Saccharides may be linked to carriers (e.g. proteins) directly [31, 32], but a linker or spacer is generally used e.g. adipic acid, β-propionamido [33], nitrophenyl-ethylamine [34], haloacyl halides [35], glycosidic linkages [36], 6-aminocaproic acid [37], ADH [38], C₄ to C₁₂ moieties [39], etc.

### Carrier Proteins in Conjugates

Typical carrier proteins in conjugates are bacterial toxins or toxoids, such as diphtheria toxoid or tetanus toxoid. The CRM₁₉₇ diphtheria toxin derivative [40-42] is the carrier protein in Menjugate™, Prevnar™ and Meningitec™, whereas tetanus toxoid is used in NeisVac™. Diphtheria toxoid is used as the carrier in Menactra™. Other known carrier proteins include the N. meningitidis outer membrane protein [43], synthetic peptides [44,45], heat shock proteins [46,47], pertussis proteins [48,49], cytokines [50], lymphokines [50], hormones [50], growth factors [50], artificial proteins comprising multiple human CD4⁺T cell epitopes from various pathogen-derived antigens [51] (e.g. N19 [52]), protein D from H. influenzae [53,54], pneumococcal surface protein PspA [55], iron-uptake proteins [56], toxin A or B from C. difficile [57], etc. Compositions may use more than one carrier protein e.g. to reduce the risk of carrier suppression, and a single carrier protein might ccarry more than one saccharide antigen [58]. Conjugates generally have a saccharide:protein ratio (w/w) of between 1:5 (i.e. excess protein) and 5:1 (i.e. excess saccharide).

### Saccharides in Conjugates

The conjugate saccharides may be polysaccharides (e.g. with a degree of polymerisation of >10, e.g. 20, 30, 40, 50, 60 or more) or oligosaccharides (e.g. with a degree of polymerisation of from about 4 to about 10). Oligosaccharides may be the result of depolymerisation and/or hydrolysis of a parent polysaccharide e.g. the analyte may be a saccharide-containing fragment of a larger saccharide. Preferred conjugate saccharides are capsular saccharides.

Even more preferred conjugate saccharides are bacterial capsular saccharides e.g. from Neisseria meningitidis (serogroups A, B, C, W135 or Y), Streptococcus pneumoniae (serotypes 4, 6B, 9V, 14, 18C, 19F, or 23F), Streptococcus agalactiae (types 1a, 1b, II, III, IV, V, VI, VII, or VIII), Haemophilus influenzae (typeable strains: a, b, c, d, e or f), Pseudomonas aeruginosa, Staphylococcus aureus, etc.

Other saccharides in conjugates can include glucans (e.g. fungal glucans, such as those in Candida albicans), and fungal capsular saccharides e.g. from the capsule of Cryptococcus neoformans. Other preferred conjugate saccharide antigens are eukaryotic saccharides e.g. fungal saccharides, plant saccharides, human saccharides (e.g. cancer antigens), etc. Other conjugate saccharides are lipopolysaccharides and lipooligosaccharides.

As well as containing saccharides, samples to be analysed can include other materials. These may or may not be retained by the chromatography column, and so may or may not be present in the eluate. Typically such components will not bind to the column.

The sample analyte may be a product to be tested prior to release (e.g. during manufacture or quality control testing), or may be a product to be tested after release (e.g. to assess stability, shelf-life, etc.).

### Vaccines

Preferred samples analysed in the present invention are vaccines comprising conjugated saccharide.

Preferred conjugate vaccines comprise immunogens protecting against:
Haemophilus influenzae type b (Hib);
Neisseria meningitidis (meningococcus) of serogroups A, C W135 and/or Y;
Streptococcus pneumoniae (pneumococcus);
Streptococcus agalactiae (group B streptococcus);
Pseudomonas aeruginosa; or
Staphylococcus aureus,
   either singly or in combination.

Preferred combination conjugate vaccines comprise:
mixtures of conjugates from each of meningococcal serogroups C and Y;
mixtures of conjugates from each of meningococcal serogroups C, W135 and Y;
mixtures of conjugates from each of meningococcal serogroups A, C, W135 and Y;
mixtures of conjugates from meningococcal serogroups A and C;
mixtures of pneumoccal conjugates;
mixed meningococcal and Hib conjugates (e.g. mixtures of Hib conjugates and conjugates from each of meningococcal serogroups A and C); or
combined meningococcal, pneumococcal and Hib conjugates.

Vaccines comprising CRM-Hib (i.e. Hib saccharide conjugated to a CRM₁₉₇ carrier) and/or CRM-MenA are particularly preferred. Other preferred vaccines are those containing:
a conjugate of diphtheria toxoid and a N. meningitidis serogroup A, C, W135 and/or Y saccharide;
a conjugate of tetanus toxoid and Hib saccharide; or
a conjugate of H. influenzae protein D and a N. meningitidis serogroup A, C, W135 and/or Y saccharide.

In addition to the conjugate, the vaccine may contain one or more of:
a protein antigen from serogroup B of N. meningitidis;
preparations of vesicles prepared from N. meningitidis serogroup B;
an antigen from hepatitis A virus, such as inactivated virus [e.g. 59, 60];
an antigen from hepatitis B virus, such as the surface and/or core antigens [e.g. 60, 61];
an antigen from Bordetella pertussis, such as pertussis holotoxin (PT) and filamentous haemagglutinin (FHA) from B. pertussis, optionally also in combination with pertactin and/or agglutinogens 2 and 3. Cellular pertussis antigens may be used instead;
a diphtheria antigen, such as a diphtheria toxoid [e.g. chapter 13 of ref. 1];
a tetanus antigen, such as a tetanus toxoid [e.g. chapter 27 of ref. 1]; or polio antigen(s), e.g. IPV.

Such antigens may be adsorbed to an aluminium salt adjuvant (e.g. a hydroxide or a phosphate). Any further saccharide antigens are preferably included as conjugates.

### Mass Spectrometry (MS) Analysis

The eluate from the ion exchange chromatography column is analysed in the present invention by MS.

It is an advantage of some embodiments of the present invention that they do not require additional on-line or off-line desalting or salt suppressing processing of the eluate prior to MS analysis. However, desalting or salt suppression treatment may optionally be performed prior to MS analysis to improve the mass spectra of the analyte. It is preferred, however, that no such desalting or salt suppression treatment is carried out before MS analysis.

A variety of MS techniques may be used in the present invention. In the spectrometer, ions may be produced by matrix-assisted laser desorption ionisation (MALDI), electrospray ionisation (ESI), or Fast-Atom Bombardment (FAB), in negative or positive modes. Preferably, ions are produced by ESI. Since it is possible to perform the invention in either positive or negative mode, a wide variety of analytes may be investigated using the invention.

The volatile ionic salt forms at least one volatile compound which evaporates from the eluate before or during ionisation in the mass spectrometer.

In the spectrometer, the mass analyser may be a time of flight (TOF), quadrupole time of flight (Q-TOF), ion trap (IT), quadrupole ion trap (Q-IT), triple quadrupole (QQQ) Ion Trap or Time-Of-Flight Time-Of-Flight (TOFTOF) or Fourier transform ion cyclotron resonance (FTICR) mass analyser. Preferably, the mass analyser is a Q-TOF mass analyser.

Preferably, the mass spectrometer is an ESI Q-TOF mass spectrometer.

The mass spectrum obtained from the MS allows the progress of fragmentation of a full-length saccharide to be checked or monitored. Furthermore, the mass spectrum obtained from the MS may be used to calculate the DP of a saccharide analyte or may be used to calculate the number of acetyl groups in a saccharide analyte, as described below. In some embodiments, therefore, the method of analysis of the invention comprises the step of determining the DP and/or the number of acetyl groups in the saccharide analyte. If desired, the mass spectrum may also be used to obtain information on the saccharide structure.

### Separation and Analysis Steps

The MS analysis of the eluate may be carried out directly after the chromatographic separation, or the eluate may be stored for a period of time prior to MS analysis. The separation and MS analysis may be carried out in the same or different locations (e.g. in different countries) by the same or different operators. Thus, the second and third aspects of the invention may be carried out independently, or combined together.

### Further Steps

For saccharide analysis, it may be desired to filter at least some non-analyte compounds from the sample before entry to the column, and Dionex™ produce pre-column traps and guards for this purpose e.g. an amino trap for removing amino acids, a borate trap, etc.

After elution and analysis, the invention may include the further step of determining a characteristic of a detected analyte e.g. its DP (typically an average DP), its molecular weight, its purity, etc.

In parallel with MS detection (preferably using a T flow split immediately after the column), the eluate may be coupled into amperometric and/or spectroscopic detectors.

### Use of the Invention in the Production and Quality Control of Vaccines

The invention may be used at several stages in the production and quality control of vaccines. The invention may be used prior to conjugation at a stage where it is necessary to ensure that correctly sized saccharide chains are selected for production of a conjugate or after conjugation for quality control of the vaccine.

The invention allows the progress of fragmentation of a full-length polysaccharide prior to conjugation to be checked or monitored. Where oligosaccharides of a particular length (or range of lengths) are desired then it is important that fragmentation of the polysaccharide should not be so extensive as to take depolymerisation past the desired point (e.g. at the extreme, to give monosaccharides). The invention allows the progress of this partial depolymerisation to be monitored, by measuring saccharide chain length over time. Thus the invention provides a process for analysing saccharide(s) in a composition, comprising the steps of: (a) starting depolymerisation of the saccharide(s) in the composition; and, at one or more time points thereafter, (b) analysing the saccharide(s) as described herein. In an initial run of experiments then it will be usual to analyse at several time points in order to determine progress over time, but after standard conditions have been established then it is usual to analyse at a set time point for confirmatory purposes. Once a desired end-point has been reached then the process may comprise the further step of: (c) stopping the depolymerisation, e.g. by washing, separating, cooling, etc. The process may also comprise the further preparative step of conjugation of the depolymerised saccharide to a carrier protein, e.g. after optional chemical activation.

The invention also allows selection of desired oligosaccharide chains after fragmentation. Thus the invention provides a process for selecting saccharides for use in preparing a glycoconjugate, comprising the steps of: (a) obtaining a composition comprising a mixture of different polysaccharide fragments; (b) separating the mixture into sub-mixtures; (c) analysing one or more sub-mixtures using a process as described herein; and (d) using the results of step (c) to select one or more sub-mixtures for use in conjugation. The process may involve fragmentation of the polysaccharide prior to step (a), or may start with an already-prepared mixture. Step (b) preferably comprises chromatographic separation. The fragments may be fragments of the same polysaccharide e.g. of the same serogroup, or of different polysaccharides, e.g. from different serogroups or species. After step (d), the process may comprise the step of conjugation to a carrier protein, e.g. after optional chemical activation.

Prior to conjugation it is usual for a saccharide to be chemically activated in order to introduce a functional group that can react with the carrier. Conditions for saccharide activation can cause hydrolysis, and so it is useful to analyse a saccharide after activation. The term “saccharide” can include either inactivated or activated saccharides, as well as poly, oligo and/or monosaccharides. Moreover, the invention provides a process for preparing an activated saccharide for use in preparing a glycoconjugate, comprising the steps of: (a) obtaining a saccharide; (b) chemically activating the saccharide to introduce a functional group that can react with a carrier protein; (c) analysing the product of step (b) as described herein; and, optionally, (d) using the results of step (c) to determine whether the saccharide is of desired size (or average size). The process may include the further step of: (e) reacting the activated saccharide, e.g. of desired size, with the carrier protein (which may itself have been activated) to give the glycoconjugate. The process may involve fragmentation of a polysaccharide prior to step (a), or may start with an already-prepared mixture.

The invention can also be used after conjugation. After conjugation, compositions can be analysed using the invention in three ways: first, total saccharides in a composition can be measured e.g. prior to mixing of different conjugates, or prior to release of a vaccine (for regulatory or quality control purposes); second, free unconjugated saccharide in a composition can be measured e.g. to check for incomplete conjugation, or to follow conjugate hydrolysis by monitoring increasing free saccharide over time; third, conjugated saccharide in a composition can be measured, for one or more of the above reasons. The first and third ways require the saccharide to be released from the conjugate prior to analysis. To separately assess conjugated and unconjugated saccharides, they must be separated. Free (i.e. unconjugated) saccharide in an aqueous composition can be separated from conjugated saccharide in various ways. The conjugation reaction changes various chemical and physical parameters for the saccharide, and the differences can be exploited for separation. For example, size separation can be used to separate free and conjugated saccharide, as the conjugated material has a higher mass due to the carrier protein. Ultrafiltration is a preferred size separation method. As a further alternative, if conjugates have been adsorbed to an adjuvant then centrifugation will separate adsorbed conjugate (pellet) from free saccharide (supernatant) that desorbs after hydrolysis.

The invention provides a method of analysing a glycoconjugate, comprising the steps of: (a) treating the glycoconjugate to release saccharide from carrier; and (b) analysing the released saccharide as described herein. The invention provides a method of analysing a glycoconjugate composition, comprising the steps of: (a) separating unconjugated saccharide in the composition from conjugated saccharide; and (b) analysing the unconjugated and/or conjugated saccharide as described above.

The invention also provides a method of releasing a vaccine for use by physicians, comprising the steps of: (a) manufacturing a vaccine, including a step of analysis as described herein; and, if the results from step (a) indicate that the vaccine is acceptable for clinical use, e.g. it has a DP or average DP acceptable for clinical use, (b) releasing the vaccine for use by physicians. Step (a) may be performed on a packaged vaccine, on a bulk vaccine prior to packaging, on saccharides prior to conjugation, etc.

The invention also provides a batch of vaccines, wherein one vaccine within the batch has been analysed using a method of the invention.

The invention also provides a method of monitoring the stability of a vaccine in storage, comprising the steps of: (a) analysing the vaccine as described herein; and, if the results from step (a) indicate that the vaccine is acceptable for clinical use, e.g. it is of suitable saccharide DP (or average DP), (b) either (i) continuing to store the vaccine or (ii) releasing the vaccine for use by physicians. Step (a) may be performed on a packaged vaccine, on a bulk vaccine prior to packaging, on saccharides prior to conjugation, etc.

The method of analysis of the invention allows the comparison of the same vaccine under different conditions, or different vaccines under the same conditions.

Thus, the invention provides a method of comparing different vaccines, comprising the steps of: (a) treating a plurality of different vaccines under substantially identical environmental conditions; (b) analysing the treated vaccines as described herein; (c) comparing the results of step (b); and, optionally, (d) selecting a vaccine, e.g. a vaccine stable under the at least one environmental condition from the plurality of different vaccines. Step (d) may, for example, comprise selecting the most stable vaccine under the at least one environmental condition. Thus, uses for this method include comparing the stability of different vaccines, e.g. under storage conditions. The environmental condition can be a chemical condition (e.g. exposure to a chemical component, e.g. a solvent, carrier etc.), pH, temperature, humidity etc. or a combination thereof. The plurality of different vaccines can typically differ in their composition, e.g. length of the saccharide, linker between the saccharide and the carrier, the carrier, presence of other vaccine components, concentration of components, excipients, adjuvants, pH, osmolarity, ionic strength etc.

The invention also provides a method of comparing the effect of different environmental conditions on a vaccine, comprising the steps of: (a) treating a plurality of substantially identical samples of a vaccine under a plurality of different environmental conditions; (b) analysing the treated samples as described herein; and (c) comparing the results of step (b); and, optionally, (d) selecting an environmental condition, e.g. an environmental condition under which the vaccine is stable from the plurality of different environmental conditions. Step (d) may, for example, comprise selecting the environmental condition under which the vaccine is most stable. Uses for this method include optimising the storage conditions of a vaccine. The environmental condition can be a chemical condition (e.g. exposure to a chemical component, e.g. a solvent, carrier etc.), pH, temperature, humidity etc. or a combination thereof.

The present invention also provides the following compositions:
(i) a composition comprising MenA saccharides having an average DP=9.6;
(ii) a composition comprising MenY saccharides having an average DP=19.6;
(iii) a composition comprising MenW saccharides having an average DP=15.6;
(iv) a composition comprising MenA saccharides having an average DP=5;
(v) a composition comprising MenA saccharides having an average DP=6;
(vi) a composition comprising MenY saccharides having an average DP=4;
(v) a composition comprising MenA saccharides having an average DP=2;
(vi) a composition comprising MenA saccharides having an average DP=4;
(vii) a composition comprising MenA saccharides having an average DP=13;
(viii) a composition comprising MenW saccharides having an average DP=3;
(ix) a composition comprising MenW saccharides having an average DP=4;
(x) a composition comprising MenW saccharides having an average DP=9;
(xi) a composition comprising MenW saccharides having an average DP=18;
(xii) a composition comprising MenY saccharides having an average DP=3;
(xiii) a composition comprising MenY saccharides having an average DP=11; and
(xiv) a composition comprising MenY saccharides having an average DP=19.

MenA saccharides are bacterial capsular saccharides from Neisseria meningitidis serogroup A.

MenW saccharides are bacterial capsular saccharides from Neisseria meningitidis serogroup W135.

MenY saccharides are bacterial capsular saccharides from Neisseria meningitidis serogroup Y.

### General

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x means, for example, x±10%.

The methods of the invention can be used for analytical and/or preparative purposes. References to “analysing”, “analysis”, etc. should not be construed as excluding preparative methods.

The degree of polymerisation (DP) of a saccharide is defined as the number of repeating units in that saccharide. For a homopolymer, the DP is thus the same as the number of monosaccharide units. For a heteropolymer, however, the DP is the number of monosaccharide units in the whole chain divided by the number of monosaccharide units in the minimum repeating unit e.g. the DP of (Glc-Gal)₁₀ is 10 rather than 20, and the DP of (Glc-Gal-Neu)₁₀ is 10 rather than 30.

### BRIEF DESCRIPTION OF DRAWINGS

FIG. 1 shows profiling of (A) a sample of MenA saccharide mixture (DP=9.6), (B) a sample of MenW saccharide mixture (DP=15.6) and (C) a sample of MenY saccharide mixture (DP=19.6).

FIG. 2 shows profiling of (A) a sample of MenA saccharide mixture (DP=9.6), (B) a sample of MenA saccharide mixture (DP=5), and (C) a sample of MenA saccharide mixture (DP=6).

FIG. 3 shows IonPac AS11 profiling of a sample of MenA saccharide mixture (DP=9.6) at different ammonium hydroxide concentrations: 50 mM (A), 10 mM (B) and 0 mM (C).

FIG. 4 shows IonPac AS11 profiling of a sample of MenY saccharide mixture (DP=19.6) at different ammonium hydroxide concentrations: 50 mM (A), 10 mM (B) and 0 mM (C).

FIG. 5 shows a single ion recording (SIR) chromatogram obtained from ESI+ of HPAEC-ZQ of two MenA saccharide mixtures (DP=9.6 and DP=6).

FIG. 6 shows a single ion recording (SIR) chromatogram obtained from ESI+ of HPAEC-ZQ of two MenY saccharide mixtures (DP=19.6 and DP=4).

FIG. 7 shows a single ion recording (SIR) chromatogram obtained from ESI− of HPAEC-ZQ of two MenA saccharide mixtures (DP=9.6 and DP=6).

FIG. 8 shows a single ion recording (SIR) chromatogram obtained from ESI− of HPAEC-ZQ of two MenY saccharide mixtures (DP=4 (A) and DP=19.6 (B)).

FIG. 9 shows a total ion current (TIC) chromatogram of HPAEC-Q-TOF of a sample of MenA saccharide mixture (DP=9.6 (A) and DP=6 (B)).

FIG. 10 shows a total ion current (TIC) chromatogram of HPAEC-Q-TOF of a sample of MenA saccharide mixture (DP=9.6) at different concentrations of ammonium hydroxide: (A) 100 mM; and (B) 50 mM.

FIG. 11 shows the spectrum of charged ions (ion mode ESI−) from a sample of MenA saccharide mixture (DP=2).

FIG. 12 shows (A) a deconvoluted mass spectrum (molecular mass) and (B) a mass spectrum of double charged ions from a sample of MenA saccharide mixture (DP=4).

FIG. 13 shows (A) a deconvoluted mass spectrum (molecular mass) and (B) a mass spectrum of double charged ions from a sample of MenA saccharide mixture (DP=6).

FIG. 14 shows (A) a deconvoluted mass spectrum (molecular mass) and (B) a mass spectrum of double charged ions from a sample of MenA saccharide mixture (DP=13).

FIG. 15 shows a total ion current (TIC) chromatogram of HPAEC-Q-TOF of a sample of MenW saccharide mixture (DP=15.6).

FIG. 16 shows (A) a deconvoluted mass spectrum (molecular mass) and (B) a mass spectrum of double charges ions from a sample of MenW saccharide mixture (DP=3).

FIG. 17 shows (A) a deconvoluted mass spectrum (molecular mass) and (B) mass spectrum of double charged ions from a sample of MenW saccharide mixture (DP=4).

FIG. 18 shows a mass spectrum of triple charged (1) and quadruple charged (2) ions from a sample of MenW saccharide mixture (DP=9).

FIG. 19 shows (A) a deconvoluted mass spectrum (molecular mass) and (B) mass spectrum of triple charged ions from a sample of MenW saccharide mixture (DP=9).

FIG. 20 shows a mass spectrum of multiple charged ions from a sample of MenW saccharide mixture (DP=18).

FIG. 21 shows (A) a deconvoluted mass spectrum (molecular mass) and (B) mass spectrum of multiple charged ions from a sample of MenW saccharide mixture (DP=18).

FIG. 22 shows a total ion current (TIC) chromatogram of HPAEC-QTOF of a sample of MenY saccharide mixture (DP=19.6) using different concentrations of ammonium hydroxide: (A) 100 mM; and (B) 50 mM.

FIG. 23 shows (A) a deconvoluted mass spectrum (molecular mass) and (B) a mass spectrum of double charged ions from a sample of MenY saccharide mixture (DP=3).

FIG. 24 shows a mass spectrum of multiple charged ions from a sample of MenY saccharide mixture (DP=11).

FIG. 25 shows (A) a deconvoluted mass spectrum (molecular mass) and (B) a mass spectrum of multiple charged ions from a sample of MenY saccharide mixture (DP=11).

FIG. 26 shows a mass spectrum of multiple charged ions from a sample of MenY saccharide mixture (DP=19).

FIG. 27 shows (A) a deconvoluted mass spectrum (molecular mass) and (B) a mass spectrum of multiple charged ions from a sample of MenY saccharide (DP=19).

FIGS. 28 to 30 show spectra from analysing a protein analyte.

### MODES FOR CARRYING OUT THE INVENTION

### Materials and Methods

The instrumentation used was:
amperometric detection: (i) BioLC Dionex DX600 equipped with ED50A, AS50, GP50
MS detection: (ii) HPLC Waters 2690+Micromass ZQ; or
   (iii) HPLC Waters 2695+Q-TOF Micro™ (Micromass)

The software employed in this instrumentation was Chromeleon™ 6.5 Dionex [(i)] and MassLynx™ 3.5 Micromass [(ii), (iii)].

The samples used were as follows:
MenA saccharide mixture, having average DP=9.6 (RS04-03-01)
MenY saccharide mixture, having average DP=19.6 (RS06-03-01)
MenW saccharide mixture, having average DP=15.6 (RS07-03-01)
MenA purified saccharide mixture, having average DP=5
MenA purified saccharide mixture, having average DP=6
MenY purified saccharide mixture, having average DP=4
MenA purified saccharide mixture, having average DP=2
MenA purified saccharide mixture, having average DP=4
MenA purified saccharide mixture, having average DP=13
MenW purified saccharide mixture, having average DP=3
MenW purified saccharide mixture, having average DP=4
MenW purified saccharide mixture, having average DP=9
MenW purified saccharide mixture, having average DP=18
MenY purified saccharide mixture, having average DP=3
MenY purified saccharide mixture, having average DP=11
MenY purified saccharide mixture, having average DP=19

The mixtures of saccharides of MenA, Y and W were diluted with MilliQ water to 1 mg/ml.

Purified saccharides were diluted with MilliQ water to about 20 μg/ml.

### DP and Total Saccharide Determination

The degree of polymerisation (DP) of the saccharide mixtures was determined by known techniques, e.g. NMR, chemical methods such as colorimetric and/or enzymatic analysis [62,63,64] or the methods described in reference 65.

The total saccharide in the saccharide mixtures was also determined by methods which are well-known in the art, e.g. by depolymerising the saccharides to give their constituent monosaccharides and analysing the saccharide content of the depolymerised monosaccharides, e.g. by HPAEC with PAD.

### Spectrum Interpretation

The mass spectra show peaks for the saccharides in the sample. These peaks may be used to calculate the DP of a saccharide in the sample.

The mass spectra may be also be used to calculate the number of acetyl groups in the saccharides, since acetyl groups are readily cleaved during ionisation and fragments of the saccharides having varying numbers of acetyl groups cleaved are observed. The mass of an acetyl group is 43 Da and therefore from an analysis of the fragments at 43 Da increments, starting from the saccharide itself leading down to the fragments with all the acetyl groups removed, it is possible to calculate the number of acetyl groups from the number of 43 Da increments. When the charge of the saccharides is not +1, but for example +2, the mass increment is 43/2 Da etc.

1. Amperometric Detection of HPAEC Output with Sodium Hydroxide Eluents

High performance anion exchange chromatography (HPAEC) coupled with pulse amperometric detection (PAD) is a common technique used for monosaccharide and polysaccharide and was used as a reference to check column performance in well-known conditions. An IonPac AS11 anion exchange column was used which is a low capacity hydroxide-selective column that allows elution of highly charged polyanions (carboxylated saccharide in MenC, W and Y and phosphorylated saccharide in MenA) at low hydroxide concentrations.

A reference profiling method (method 1) utilised the IonPac AS11 column and a AG11 Guard column combined with linear gradient elution of 35 minutes from 4 mM to 100 mM NaOH at a flow rate of 1.0 ml/min. Detection was performed using the waveform for carbohydrates with triple potential, Ag/AgCl as reference.

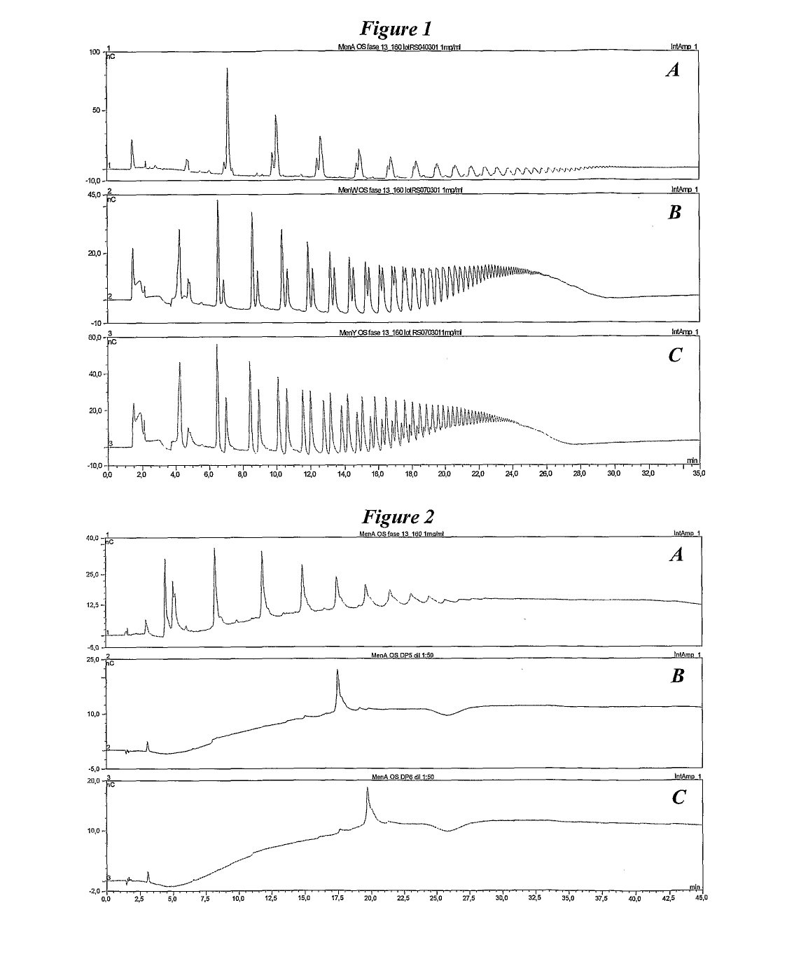

FIG. 1 shows profiling by method 1 of (A) a sample of MenA saccharide mixture (DP=9.6), (B) a sample of MenW saccharide mixture (DP=15.6) and (C) a sample of MenY saccharide mixture (DP=19.6).

2. Amperometric Detection of HPAEC Output with Ammonium Buffer Eluents.

Chromatographic separation using ammonium salt eluents was optimised by replacing the sodium hydroxide eluent of method 1 with a ammonium hydroxide/ammonium acetate eluent (method 2). A IonPac AS11+AG11 Guard column was also utilised and combined with a linear gradient elution of 40 minutes from 100 mM to 1000 mM NH₄OAc/200 mM NH₄OH at a flow rate of 1.0 ml/mon. Detection was performed using the waveform for carbohydrates with triple potential, Ag/AgCl as reference.

FIG. 2 shows profiling by method 2 of (A) a sample of MenA saccharide mixture (DP=9.6), (B) a sample of MenA saccharide mixture (DP=5), and (C) a sample of MenA saccharide mixture (DP=6).

3. Amperonzetric Detection of HPAEC Output with Ammonium Buffer Eluents Optimised for Different Antigens

The separation of method 2 was combined with ammonium acetate/hydroxide gradient elution optimised for different antigens as follows (method 3):

### MenA Analysis:

(i) 2 minutes at 20 mM NH₄OAc/50 mM NH₄OH
(ii) linear gradient of 30 minutes from 20 mM to 320 mM NH₄OAc/50 mM NH₄OH
(iii) linear gradient of 20 minutes from 320 mM to 640 mM NH₄OAc/50 mM NH₄OH
(iv) linear gradient of 10 minutes from 640 mM to 840 mM NH₄OAc/50 mM NH₄OH
(v) 13 minutes at 840 mM NH₄OAc/50 mM NH₄OH flow rate of 1.0 ml/min
detection was performed using the waveform for carbohydrates with triple potential, Ag/AgCl as reference after post-column addition of sodium hydroxide 0.5M at flow rate of 0.4 ml/min.

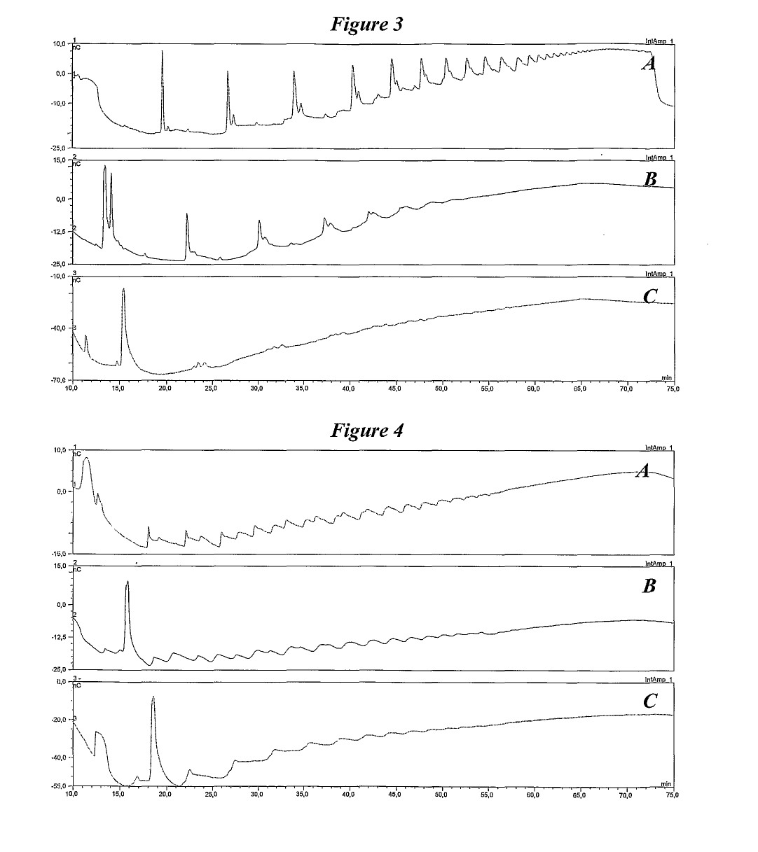

FIG. 3 shows IonPac AS11 profiling by method 3 of a sample of MenA saccharide mixture (DP=9.6) at different ammonium hydroxide concentrations: 50 mM (A), 10 mM (B) and 0 mM (C).

### MenY and MenW Analysis:

(i) 2 minutes at 20 mM NH₄OAc/50 mM NH₄OH
(ii) linear gradient of 50 minutes from 20 mM to 320 mM NH₄OAc/50 mM NH₄OH
(iii) linear gradient of 32 minutes from 320 mM to 640 mM NH₄OAc/50 mM NH₄OH
(iv) linear gradient of 10 minutes from 640 mM to 840 mM NH₄OAc/50 mM NH₄OH
(v) 10 minutes at 840 mM NH₄OAc/50 mM NH₄OH flow rate of 1.0 ml/min
detection was performed using the waveform for carbohydrates with triple potential, Ag/AgCl as reference after post-column addition of sodium hydroxide 0.5M at flow rate of 0.4 ml/min.

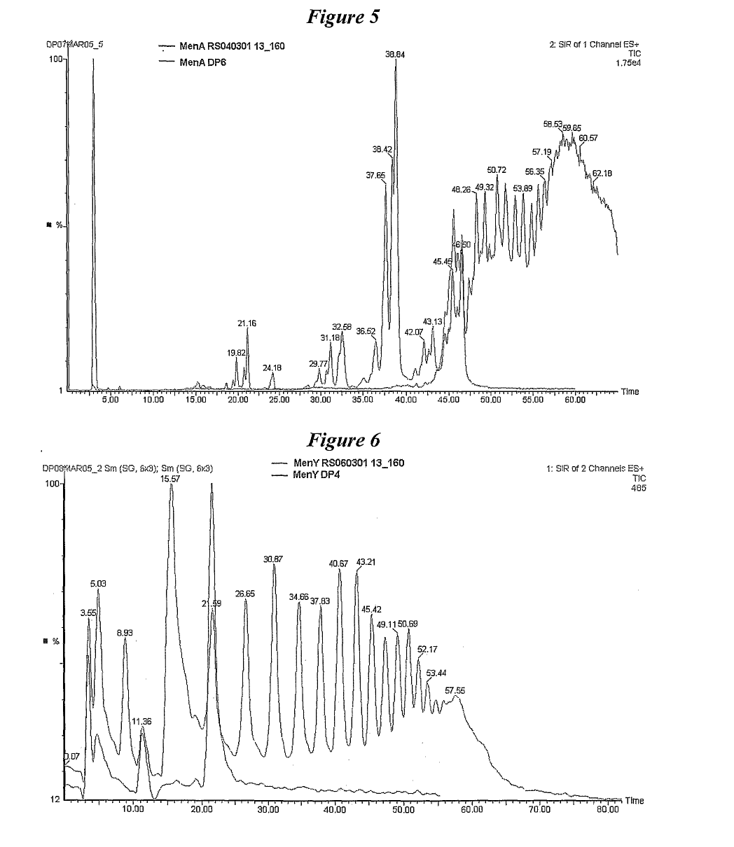

FIG. 4 shows IonPac AS11 profiling by method 3 of a sample of MenY saccharide mixture (DP=19.6) at different ammonium hydroxide concentrations: 50 mM (A), 10 mM (B) and 0 mM (C).

It can be observed that amperometric detection has some difficulties due to loss of hydroxide, however the column maintains its resolution capability.

4. MS Detection (ZQ 4000) of HPAEC Output with Ammonium Buffer Eluents Optimised for Different Antigens

The new chromatographic conditions of the invention were tested using a single-quadrupole mass spectrometer (ZQ 4000) in ESI+ (method 4) and ESI− (method 5) modes:

### Method 4

Method 4 utilised an IonPac AS11+AG11 Guard column combined with ammonium acetate/hydroxide gradient elution optimised for the different antigens.

### MenA Analysis:

(i) linear gradient of 30 minutes from 20 mM to 320 mM NH₄OAc/10% acetonitrile (ACN)
(ii) linear gradient of 20 minutes from 320 mM to 640 mM NH₄OAc/10% ACN
(iii) linear gradient of 10 minutes from 640 mM to 840 mM NH₄OAc/10% ACN
(iv) 5 minutes at 840 mM NH₄OAc/10% ACN flow rate of 0.5 ml/min
detection was performed using spectrometer ZQ 4000 (scan: source ESI+, capillary 3.0 kV, cone 30V, mass range 250-3000 m/z; SIR: source ESI+, cone 80V, mass 326 m/z).

FIG. 5 shows a single ion recording (SIR) chromatogram obtained by method 4 from ESI+ of HPAEC-ZQ of two MenA saccharide mixtures (DP=9.6 and DP=6).

### MenY and MenW Analysis:

(i) linear gradient of 50 minutes from 20 mM to 320 mM NH₄OAc/10% ACN
(ii) linear gradient of 32 minutes from 320 mM to 640 mM NH₄OAc/10% ACN flow rate of 0.5 ml/min
detection was performed using spectrometer ZQ 4000 (scan: source ESI+, capillary 3.0 kV, cone 30V, mass range 250-3000 m/z; SIR: source ESI+, cone 80V, mass 326 m/z).

FIG. 6 shows a single ion recording (SIR) chromatogram obtained by method 4 from ESI+ of HPAEC-ZQ of two MenY saccharide mixtures (DP=19.6 and DP=4).

### Method 5

Method 5 utilised an IonPac AS11+AG11 Guard column combined with ammonium acetate/hydroxide gradient elution optimised for the different antigens.

### MenA Analysis:

(i) linear gradient of 30 minutes from 20 mM to 320 mM NH₄OAc/50 mM NH₄OH/10% ACN
(ii) linear gradient of 20 minutes from 320 mM to 640 mM NH₄OAc/50 mM NH₄OH/10% ACN
(iii) linear gradient of 10 minutes from 640 mM to 840 mM NH₄OAc/50 mM NH₄OH/10% ACN
(iv) 5 minutes at 840 mM NH₄OAc/50 mM NH₄OH/10% ACN flow rate of 0.5 ml/min
detection was performed using spectrometer ZQ 4000 (scan: source ESI−, capillary 3.0 kV, cone 30V, mass range 250-3000 m/z; SIR: source ESI−, cone 80V, mass 324 m/z)

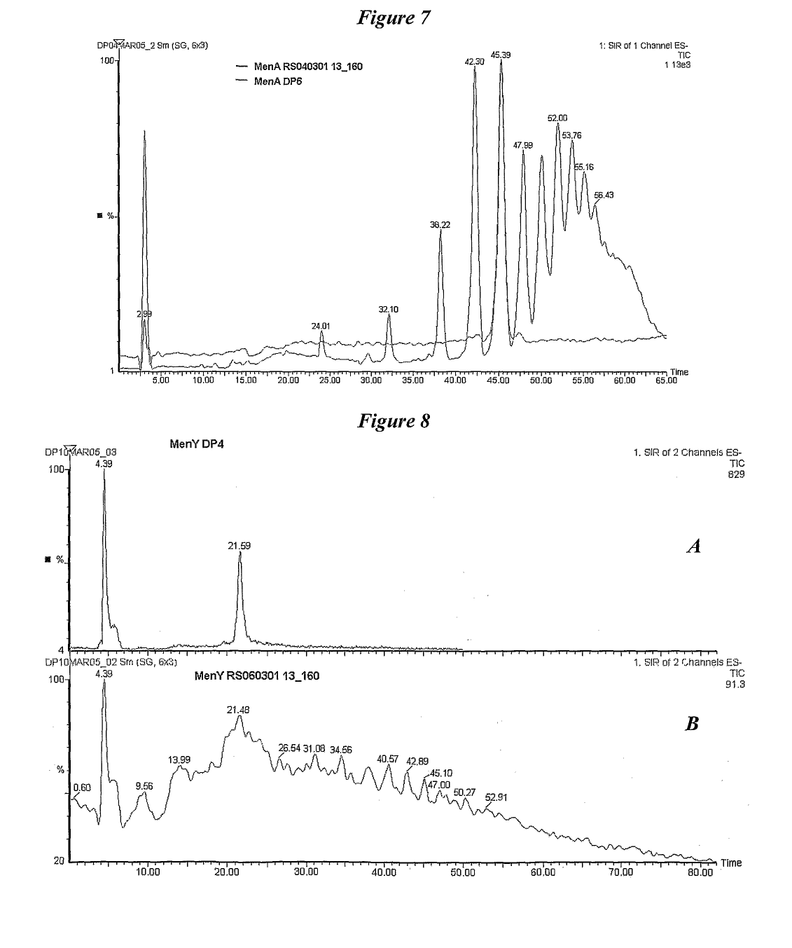

FIG. 7 shows a single ion recording (SIR) chromatogram obtained by method 5 from ESI− of HPAEC-ZQ of two MenA saccharide mixtures (DP=9.6 and DP=6).

### MenY and MenW Analysis:

(i) linear gradient of 50 minutes from 20 mM to 320 mM NH₄OAc/50 mM NH₄OH/10% ACN
(ii) linear gradient of 32 minutes from 320 mM to 640 mM NH₄OAc/50 mM NH₄OH/10% ACN flow rate of 0.5 ml/min
detection was performed using spectrometer:
ZQ 4000 (scan: source ESI−, capillary 3.0 kV, cone 30V, mass range 250-3000 m/z; SIR: source ESI−, cone 80V, mass 452 and 470 m/z)

FIG. 8 shows a single ion recording (SIR) chromatogram obtained by method 5 from ESI− of HPAEC-ZQ of two MenY saccharide mixtures (DP=4 (A) and DP=19.6 (B)).

5. MS Detection (Q-TOF) of HPAEC Output with Ammonium Buffer Eluents Optimised for Different Antigens

The new chromatographic conditions of the invention were tested using a Q-TOF mass spectrometer in ESI− mode (method 6). This method utilised an IonPac AS11+AG11 Guard column combined with ammonium acetate/hydroxide gradient elution optimised for the different antigens.

### MenA Analysis:

(i) linear gradient of 30 minutes from 20 mM to 320 mM NH₄OAc/50 mM NH₄OH/10% ACN
(ii) linear gradient of 20 minutes from 320 mM to 640 mM NH₄OAc/50 mM NH₄OH/10% ACN
(iii) linear gradient of 10 minutes from 640 mM to 840 mM NH₄OAc/50 mM NH₄OH/10% ACN
(iv) 5 minutes at 840 mM NH₄OAc/50 mM NH₄OH/10% ACN flow rate of 0.5 ml/min
detection was performed using Q-TOF spectrometer (source ESI−, capillary 3.0 kV, cone 30V, mass range 200-3000 m/z)

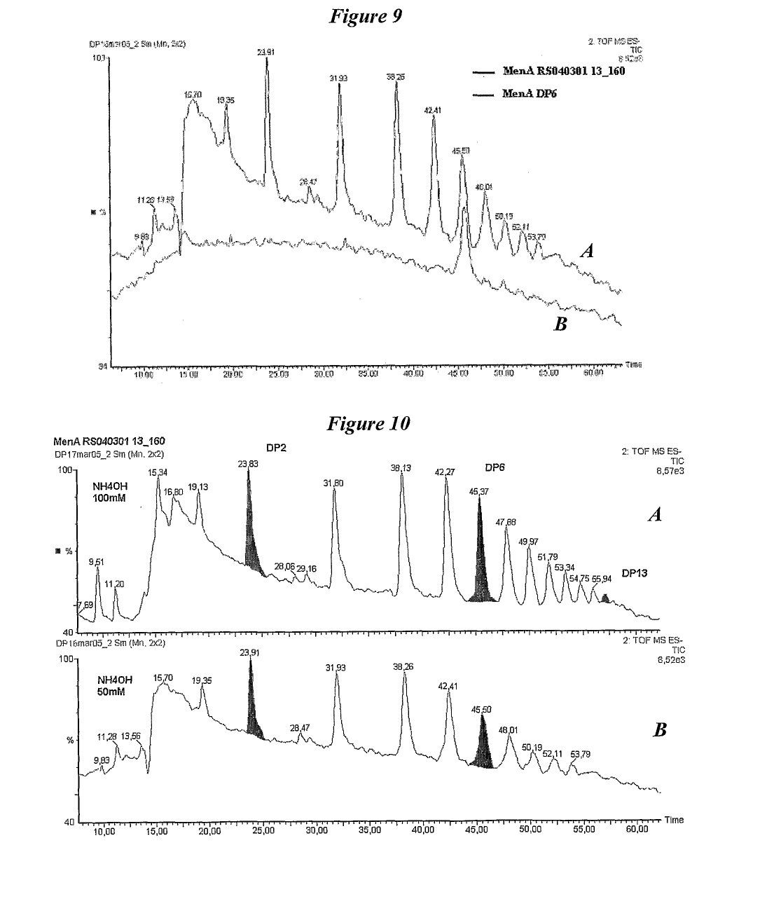

FIG. 9 shows a total ion current (TIC) chromatogram of HPAEC-Q-TOF by method 6 of a sample of MenA saccharide mixture (DP=9.6).

FIG. 10 shows a total ion current (TIC) chromatogram of HPAEC-Q-TOF by method 6 of a sample of MenA saccharide mixture (DP=9.6) at different concentrations of ammonium hydroxide: (A) 100 mM; and (B) 50 mM.

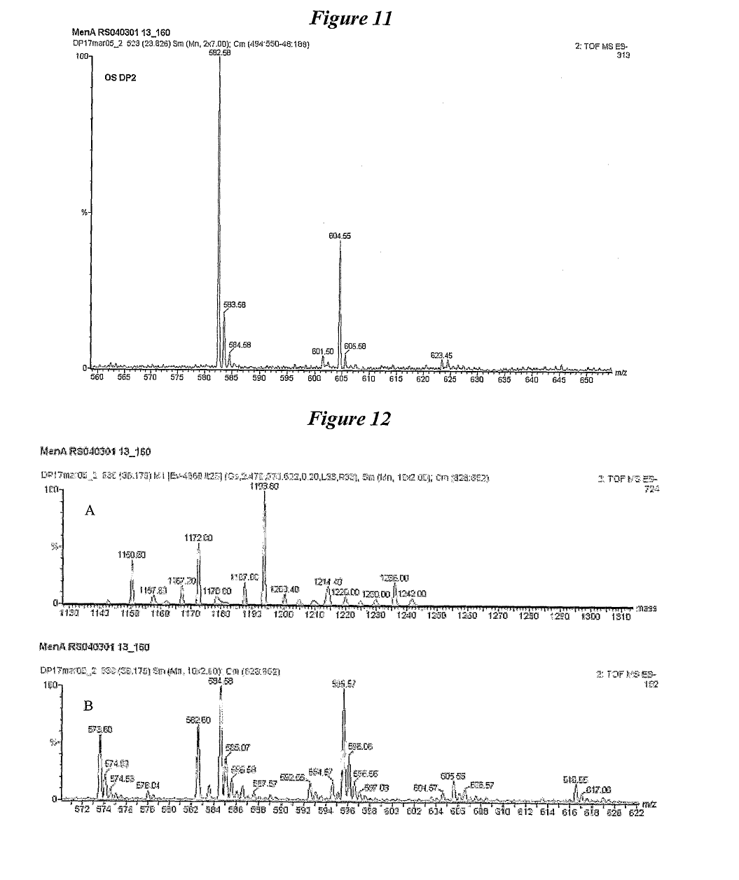

FIG. 11 shows the spectrum of charged ions by method 6 (ion mode ESI−) from a sample of MenA saccharide mixture (DP=2). Table 1 shows the theoretical and observed m/z ions from a sample of MenA saccharide mixture (DP=2).

TABLE 1
MenA OS DP2

| Observed ions | Expected ions | O-acetyl | Na | Charge |
|---|---|---|---|---|
| 582.58 | 583.09 | 0 | 0 | 1 |
| 604.55 | 605.08 | 0 | 1 | 1 |
| 623.45 | 625.10 | 1 | 0 | 1 |

FIG. 12 shows (A) a deconvoluted mass spectrum (molecular mass) and (B) a mass spectrum of double charged ions from a sample of MenA saccharide mixture (DP=4) by method 6. Table 2 shows the theoretical and observed mass and m/z ions from a sample of MenA saccharide mixture (DP=4).

TABLE 2
MenA OS DP4

| Observed | Expected | | | Observed | Expected | |
|---|---|---|---|---|---|---|
| mass | mass | O-acetyl | Na | ions | ions | Charge |
| 1150.60 | 1150.19 | 0 | 0 | 573.60 | 574.10 | 2 |
| 1172.80 | 1172.18 | 0 | 1 | 584.58 | 585.09 | 2 |
| 1193.80 | 1194.16 | 0 | 2 | 595.57 | 596.08 | 2 |
| 1214.40 | 1214.19 | 1 | 1 | 605.56 | 606.09 | 2 |
| 1236.00 | 1236.17 | 1 | 2 | 616.55 | 617.08 | 2 |

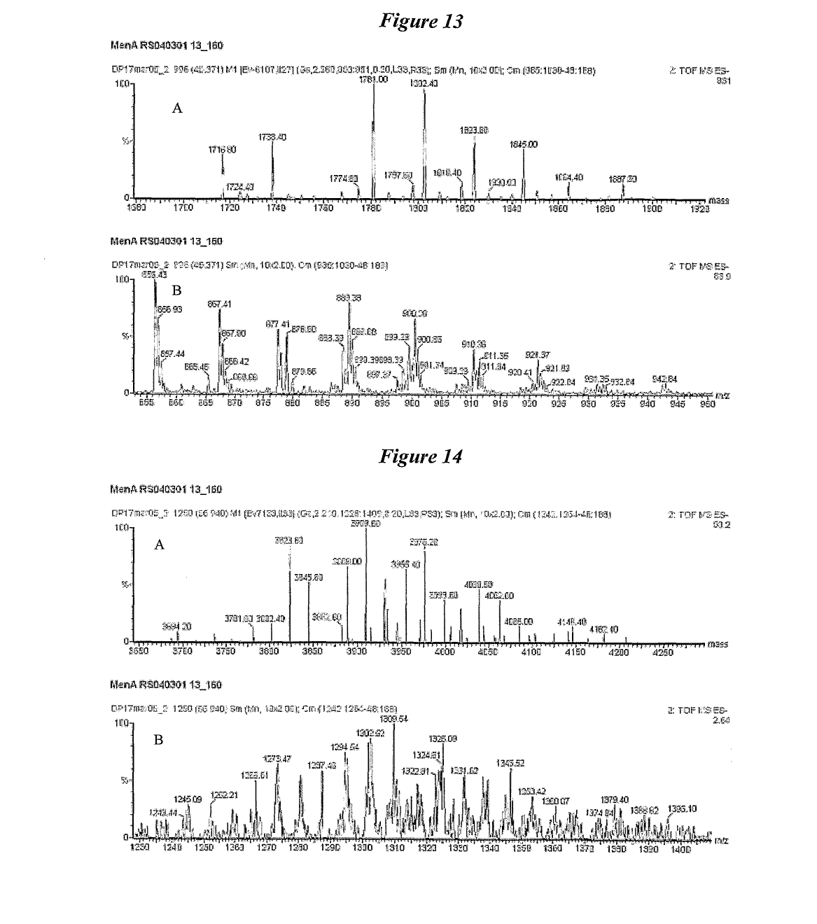

FIG. 13 shows (A) a deconvoluted mass spectrum (molecular mass) and (B) a mass spectrum of double charged ions from a sample of MenA saccharide mixture (DP=6) by method 6. Table 3 shows the theoretical and observed mass and m/z ions from a sample of MenA saccharide mixture (DP=6).

TABLE 3
MenA OS DP6

| Observed | Expected | | | Observed | Expected | |
|---|---|---|---|---|---|---|
| mass | mass | O-acetyl | Na | ions | ions | Charge |
| 1716.80 | 1716.28 | 0 | 0 | 856.43 | 857.14 | 2 |
| 1738.40 | 1738.26 | 0 | 1 | 867.41 | 868.13 | 2 |
| — | 1760.24 | 0 | 2 | 878.90 | 879.12 | 2 |
| 1781.00 | 1780.27 | 1 | 1 | 889.38 | 889.13 | 2 |
| 1802.40 | 1802.25 | 1 | 2 | 900.36 | 900.13 | 2 |
| 1823.80 | 1822.28 | 2 | 1 | 910.36 | 910.14 | 2 |
| 1845.00 | 1844.26 | 2 | 2 | 921.37 | 921.13 | 2 |
| 1864.40 | 1864.29 | 3 | 1 | 931.35 | 931.15 | 2 |
| 1887.60 | 1886.27 | 3 | 2 | 942.84 | 942.14 | 2 |

FIG. 14 shows (A) a deconvoluted mass spectrum (molecular mass) and (B) a mass spectrum of double charged ions from a sample of MenA saccharide mixture (DP=13) by method 6.

### MenY and MenW Analysis:

(i) linear gradient of 50 minutes from 20 mM to 320 mM NH₄OAc/50 mM NH₄OH/10% ACN
(ii) linear gradient of 32 minutes from 320 mM to 640 mM NH₄OAc/50 mM NH₄OH/10% ACN flow rate of 0.5 ml/min
detection was performed using QTOF spectrometer (source ESI−, capillary 3.0 kV, cone 30V, mass range 200-3000 m/z)

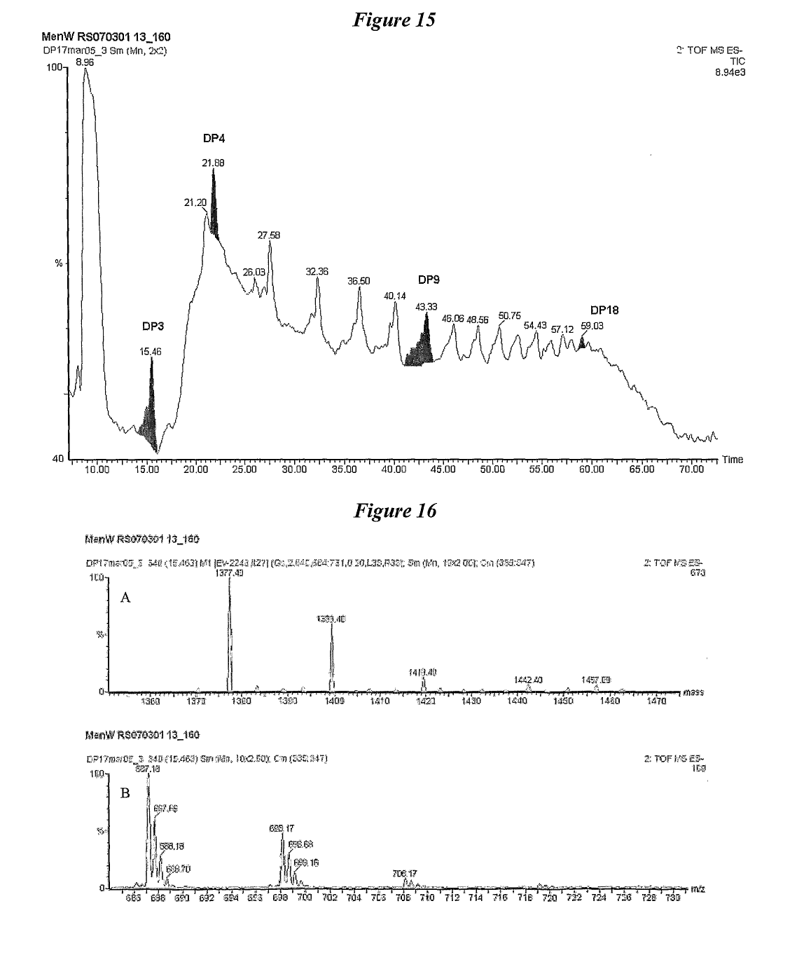

FIG. 15 shows a total ion current (TIC) chromatogram of HPAEC-Q-TOF by method 6 of a sample of MenW saccharide mixture (DP=15.6).

FIG. 16 shows (A) a deconvoluted mass spectrum (molecular mass) and (B) a mass spectrum of double charges ions from a sample of MenW saccharide mixture (DP=3) by method 6. Table 4 shows the theoretical and observed mass and m/z ions from a sample of MenW saccharide mixture (DP=3).

TABLE 4
MenW OS DP3

| Observed | Expected | | | Observed | Expected | |
|---|---|---|---|---|---|---|
| mass | mass | O-acetyl | Na | ions | ions | Charge |
| 1377.40 | 1377.46 | 0 | 0 | 687.18 | 687.73 | 2 |
| 1399.40 | 1399.44 | 0 | 1 | 698.17 | 698.72 | 2 |
| 1419.40 | 1421.42 | 0 | 2 | 708.17 | 709.71 | 2 |
| 1442.40 | 1441.45 | 1 | 1 | 719.13 | 719.72 | 2 |

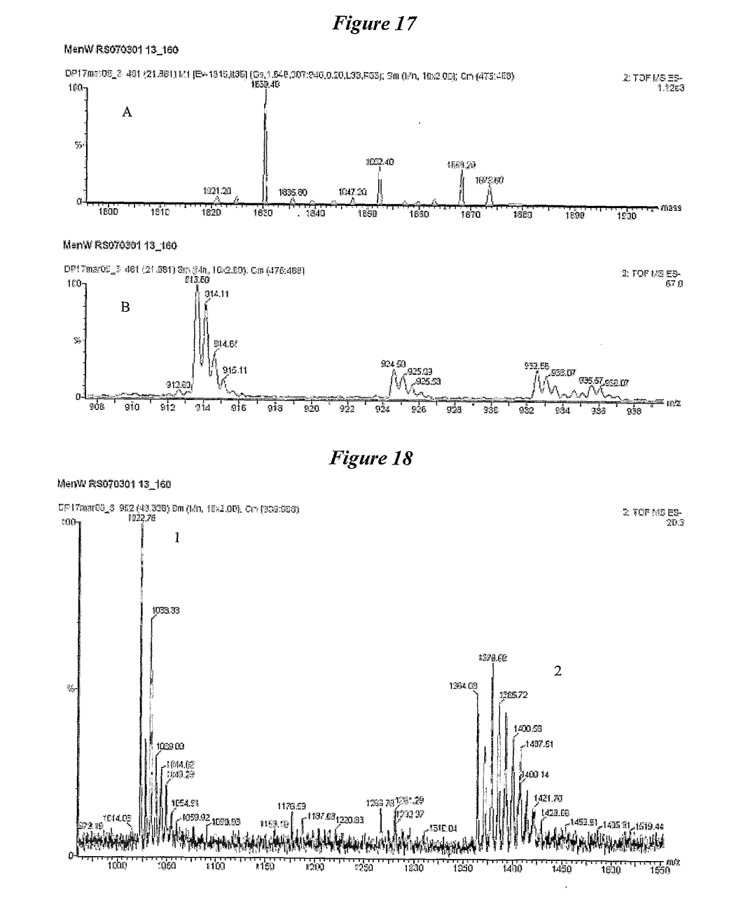

FIG. 17 shows (A) a deconvoluted mass spectrum (molecular mass) and (B) mass spectrum of double charged ions from a sample of MenW saccharide mixture (DP=4) by method 6. Table 5 shows the theoretical and observed mass and m/z ions from a sample of MenW saccharide mixture (DP=4).

TABLE 5
MenW OS DP4

| Observed | Expected | | | Observed | Expected | |
|---|---|---|---|---|---|---|
| mass | mass | O-acetyl | Na | ions | ions | Charge |
| 1830.40 | 1830.60 | 0 | 0 | 913.60 | 914.30 | 2 |
| 1852.40 | 1852.59 | 0 | 1 | 924.58 | 925.29 | 2 |
| 1873.60 | 1874.57 | 0 | 2 | 935.57 | 936.28 | 2 |

FIG. 18 shows a mass spectrum of triple charged (1) and quadruple charged (2) ions from a sample of MenW saccharide mixture (DP=9) by method 6.

FIG. 19 shows (A) a deconvoluted mass spectrum (molecular mass) and (B) mass spectrum of triple charged ions from a sample of MenW saccharide mixture (DP=9) by method 6.

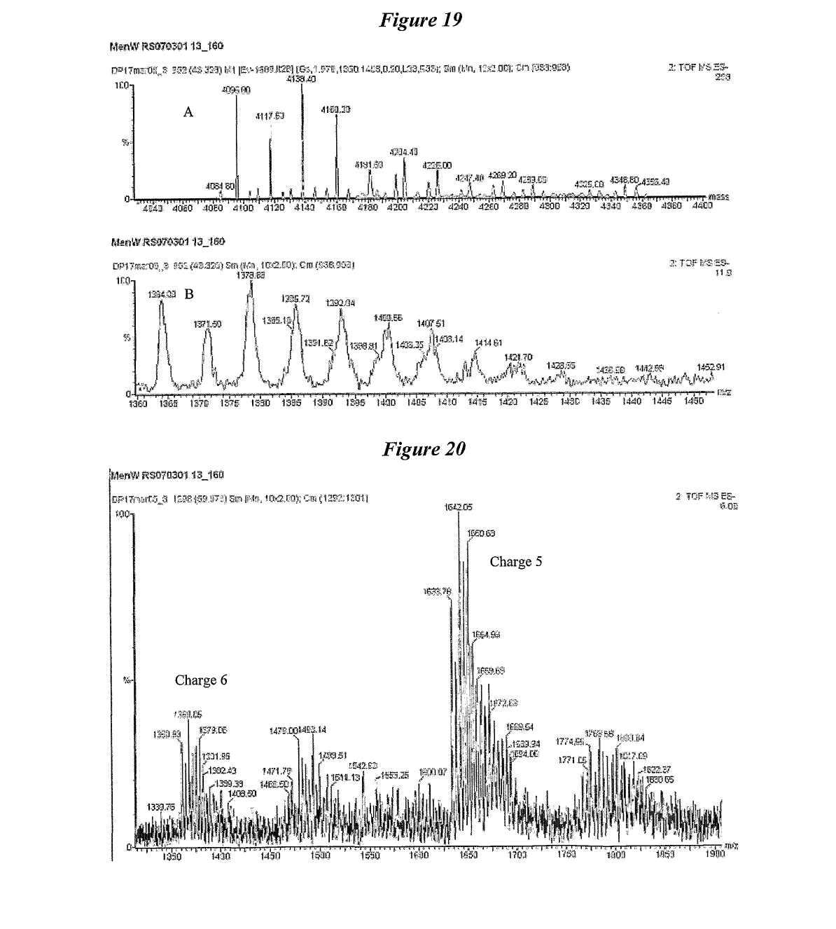

FIG. 20 shows a mass spectrum of multiple charged ions from a sample of MenW saccharide mixture (DP=18) by method 6.

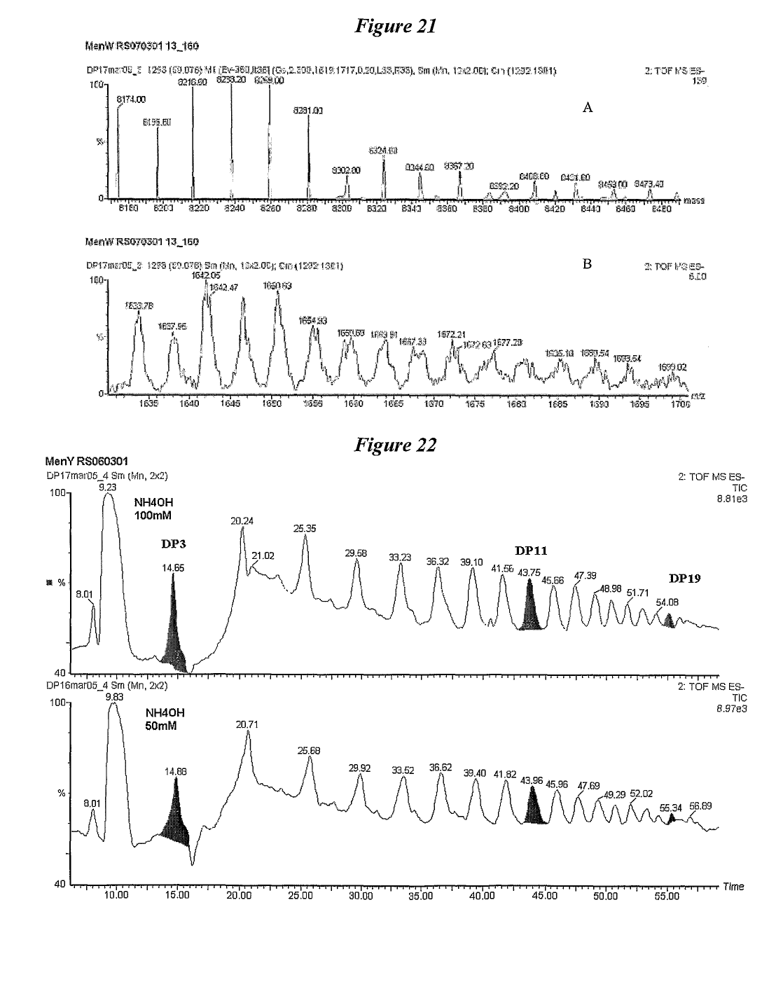

FIG. 21 shows (A) a deconvoluted mass spectrum (molecular mass) and (B) mass spectrum of multiple charged ions from a sample of MenW saccharide mixture (DP=18) by method 6.

FIG. 22 shows a total ion current (TIC) chromatogram of HPAEC-QTOF by method 6 of a sample of MenY saccharide mixture (DP=19.6) using different concentrations of ammonium hydroxide: (A) 100 mM; and (B) 50 mM.

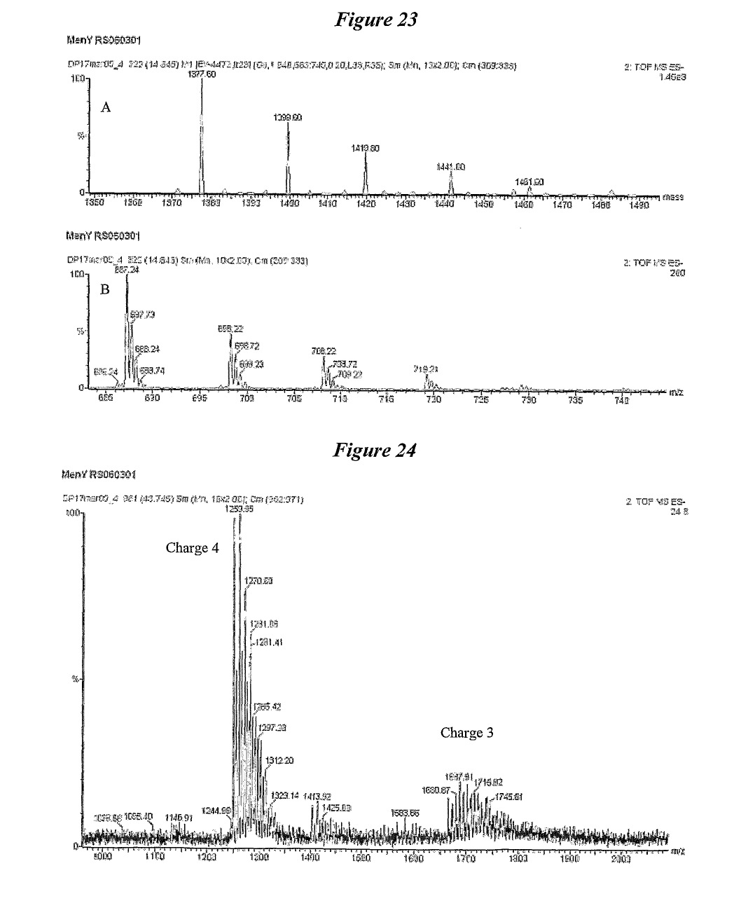

FIG. 23 shows (A) a deconvoluted mass spectrum (molecular mass) and (B) a mass spectrum of double charged ions from a sample of MenY saccharide mixture (DP=3) by method 6. Table 6 shows the theoretical and observed mass and m/z ions for a sample of MenY saccharide mixture (DP=3).

TABLE 6
MenY OS DP3

| Observed | Expected | | | Observed | Expected | |
|---|---|---|---|---|---|---|
| mass | mass | O-acetyl | Na | ions | ions | Charge |
| 1377.60 | 1377.46 | 0 | 0 | 687.24 | 687.73 | 2 |
| 1399.60 | 1399.44 | 0 | 1 | 698.22 | 698.72 | 2 |
| 1419.80 | 1421.42 | 0 | 2 | 708.22 | 709.71 | 2 |
| 1441.60 | 1441.45 | 1 | 1 | 719.21 | 719.72 | 2 |

FIG. 24 shows a mass spectrum of multiple charged ions from a sample of MenY saccharide mixture (DP=11) by method 6.

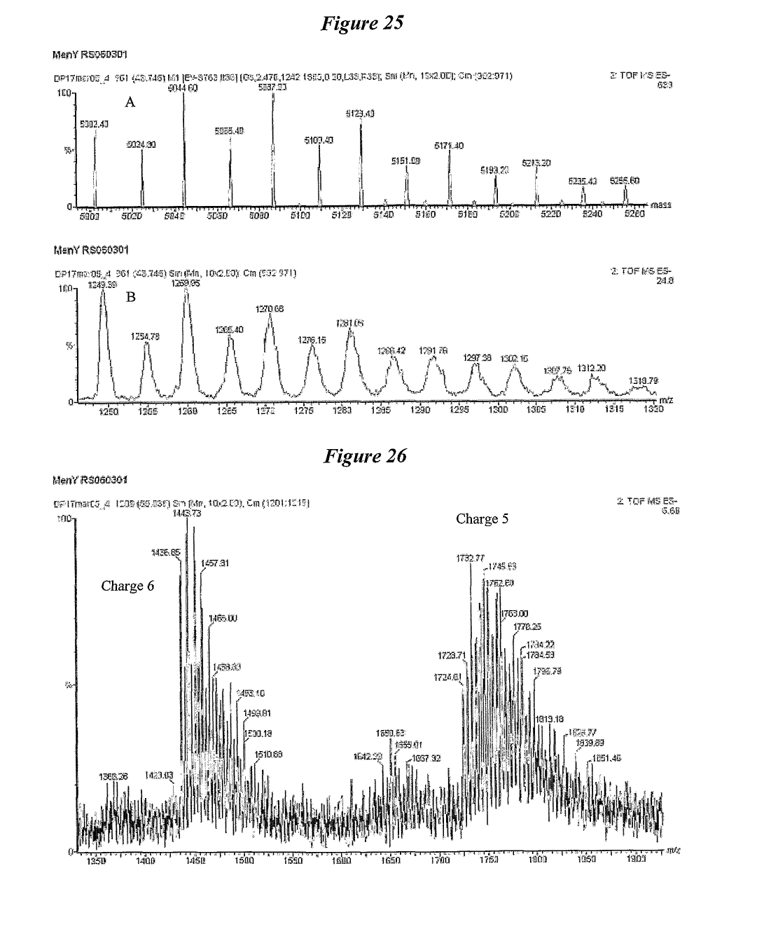

FIG. 25 shows (A) a deconvoluted mass spectrum (molecular mass) and (B) a mass spectrum of multiple charged ions from a sample of MenY saccharide mixture (DP=11) by method 6.

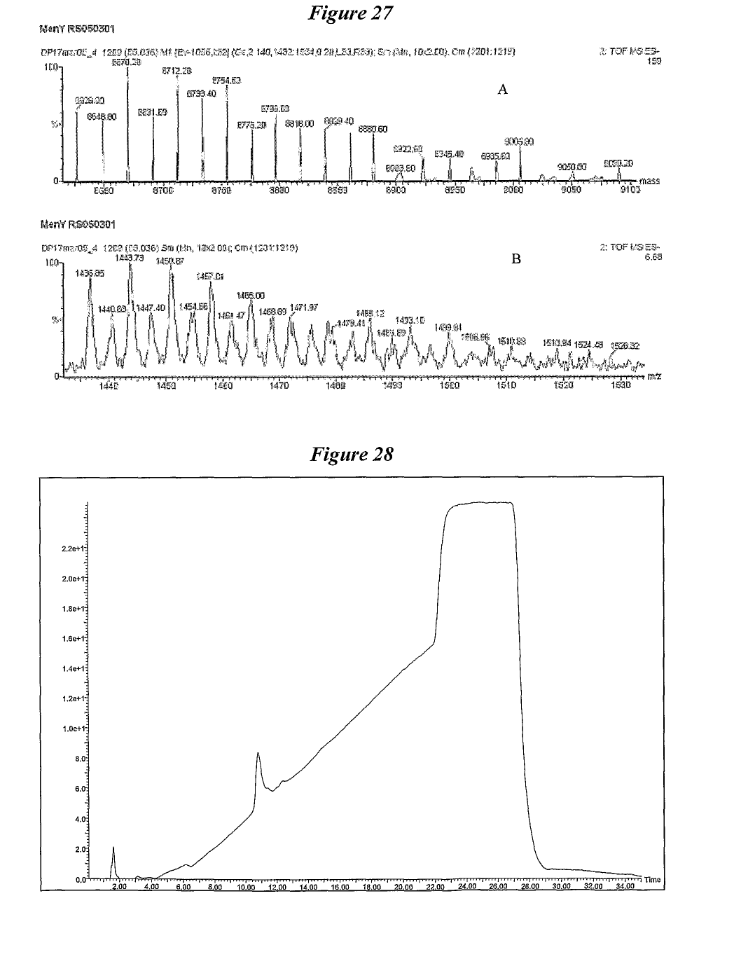

FIG. 26 shows a mass spectrum of multiple charged ions from a sample of MenY saccharide mixture (DP=19) by method 6.

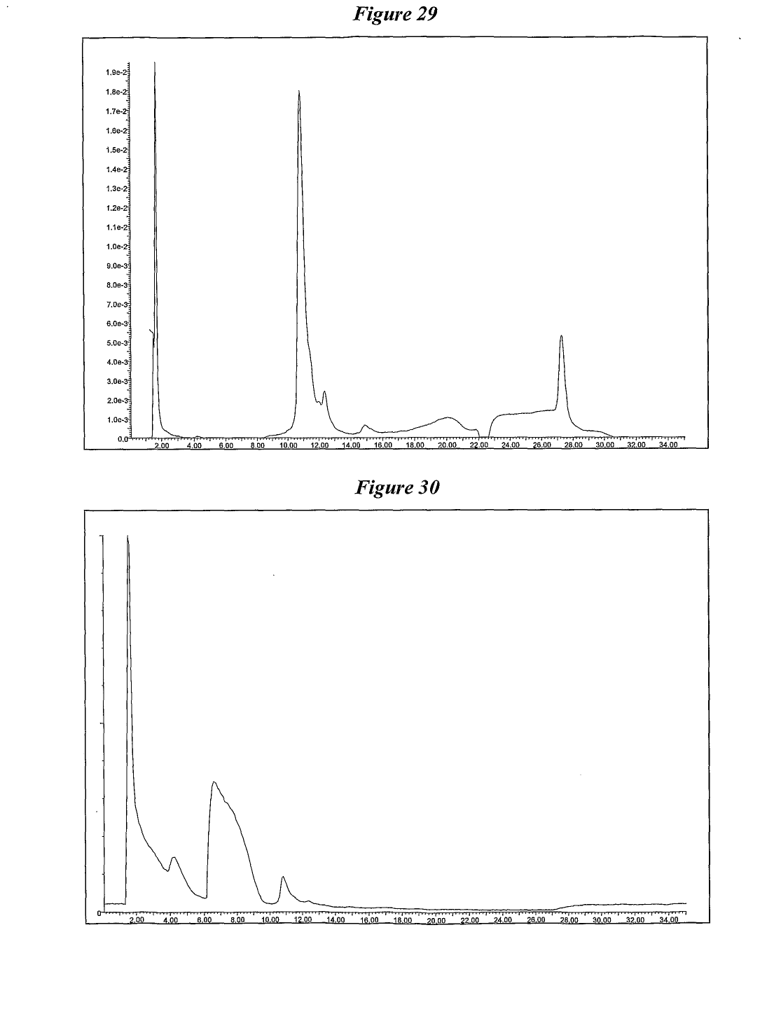

FIG. 27 shows (A) a deconvoluted mass spectrum (molecular mass) and (B) a mass spectrum of multiple charged ions from a sample of MenY saccharide (DP=19) by method.

### Conclusions

It can be seen that the invention can provide direct interface of HPAEC with mass spectrometry allowing information concerning the chemical structure and composition of a sample to be analysed, while avoiding off-line or on-line sample treatment prior to MS analysis. With appropriate standards (e.g. of single oligosaccharides) the concentration of saccharide in a sample may also be analysed using the invention.

6. MS Detection of HPAEC Output with Ammonium Acetate Eluent for Protein Analyte

A fusion protein of two meningococcal proteins was purified by HPAEC with direct coupling to mass spectrometry to measure its molecular mass without any out-line work.

A ProPac WAX-10, 4×250 mm, column from Dionex was used. The injection volume was 50 μl. Two eluents were used: (A) water and 0.1% HCOOH; (B) 1M ammonium acetate+0.1% HCOOH. These were applied with the following gradient:

| Time | A % | B % |
|---|---|---|
| 0.00 | 85.0 | 15.0 |
| 20.00 | 50.0 | 50.0 |
| 20.10 | 0.0 | 100.0 |
| 25.00 | 0.0 | 100.0 |
| 25.10 | 85.0 | 15.0 |
| 35.00 | 85.0 | 15.0 |

The mass spectrometry used polarity ES+, a 3000V capillary, a 30V sample cone, a 0.5V extraction cone, a desolvation temperature of 150° C., a source temperature of 100° C., a mass range of 500-5000, a 35 minute run time with a 5 second scan time and a 0.1 second interscan time.

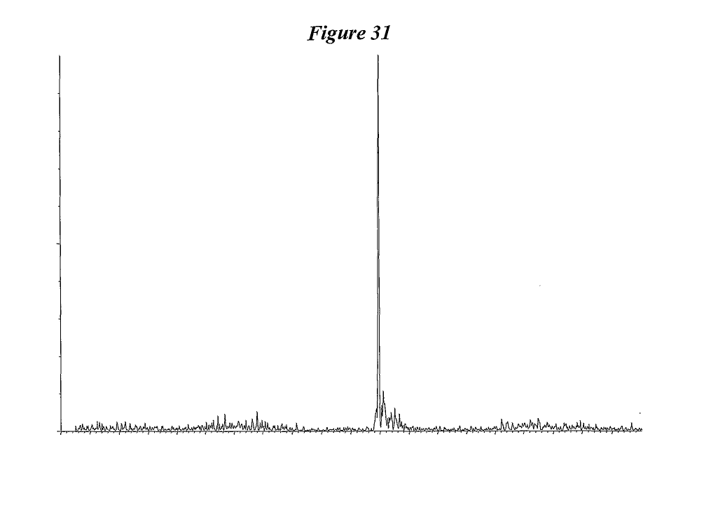

FIGS. 28 to 30 show HPAEC plots. FIG. 31 shows TOF MS for the m/z range 65-70,000, with the main peak at 67738.

It will be understood that the invention has been described by way of example only and modifications may be made whilst remaining within the scope and spirit of the invention.

### REFERENCES

### The Contents of which are Hereby Incorporated by Reference

[1] Vaccines (eds. Plotkin et al.) 4th edition, ISBN: 0721696880.
[2] Baker et al. (2003) J Infect Dis 188:66-73.
[3] Theilacker et al. (2003) Infect Immun 71:3875-84.
[4] Anonymous (2003) Drugs R D 4:383-5.
[5] Jones (2001) Curr Opin Investig Drugs 2:47-49.
[6] WO02/058737.
[7] WO03/007985.
[8] Rennels et al. (2002) Pediatr Infect Dis J21:978-979.
[9] Campbell et al. (2002) J Infect Dis 186:1848-1851.
[10] Costantino et al. (1992) Vaccine 10:691-698.
[11] Lieberman et al. (1996) JAMA 275:1499-1503.
[12] Darkes & Plosker (2002) Paediatr Drugs 4:609-630.
[13] Ugozzoli (2002) J Infect Dis 186:1358-61.
[14] Granoff et al. (1997) Infect Immun 65:1710-5.
[15] Paradiso & Lindberg (1996) Dev Biol Stand 87:269-275.
[16] Hardy et al. (1988) Anal Biochem 170:54-62.
[17] Wang et al. (1990) Anal Biochem 190:182-187.
[18] Jones (2001) Curr Opin Investig Drugs 2:47-49.
[19] Corbel (1996) Dev Biol Stand 87:113-124.
[20] WO03/080678.
[21] Ramsay et al. (2001) Lancet 357(9251):195-196.
[22] Lindberg (1999) Vaccine 17 Suppl 2:S28-36.
[23] Buttery & Maxon (2000) J R Coll Physicians Lond 34:163-168.
[24] Ahmad & Chapnick (1999) Infect Dis Clin North Am 13:113-133, vii.
[25] Goldblatt (1998) J. Med. MicrobioL 47:563-567.
[26] European patent 0477508.
[27] U.S. Pat. No. 5,306,492.
[28] WO98/42721.
[29] Conjugate Vaccines (eds. Cruse et al.) ISBN 3805549326, particularly vol. 10:48-114.
[30] Hermanson (1996) Bioconjugate Techniques ISBN: 0123423368 or 012342335X.
[31] U.S. Pat. No. 4,761,283
[32] U.S. Pat. No. 4,356,170
[33] WO00/10599
[34] Geyer et al. Med. Microbial. Immunol, 165: 171-288 (1979).
[35] U.S. Pat. No. 4,057,685.
[36] U.S. Pat. Nos. 4,673,574; 4,761,283; 4,808,700.
[37] U.S. Pat. No. 4,459,286.
[38] U.S. Pat. No. 4,965,338
[39] U.S. Pat. No. 4,663,160.
[40] Anonymous (January 2002) Research Disclosure, 453077.
[41] Anderson (1983) Infect Immun 39(1):233-238.
[42] Anderson et al. (1985) J Clin Invest 76(1):52-59.
[43] EP-A-0372501.
[44] EP-A-0378881.
[45] EP-A-0427347.
[46] WO93/17712
[47] WO94/03208.
[48] WO98/58668.
[49] EP-A-0471177.
[50] WO91/01146
[51] Falugi et al. (2001) Eur J Immunol 31:3816-3824.
[52] Baraldo et al, (2004) Infect Immun. 72:4884-7
[53] EP-A-0594610.
[54] WO00/56360.
[55] WO02/091998.
[56] WO01/72337
[57] WO00/61761.
[58] WO99/42130
[59] Bell (2000) Pediatr Infect Dis J 19:1187-1188.
[60] Iwarson (1995) APMIS 103:321-326.
[61] Gerlich et al. (1990) Vaccine 8 Suppl:S63-68 & 79-80.
[62] Ravenscroft et al. (1999) Vaccine 17:2802-2816
[63] Costantino et al. (1999) Vaccine 17:1251-1263
[64] D'Ambra et al. (1997) Anal Biochem 250:228-236
[65] WO2005/113607

1. (canceled)

2. A method of analysing a sample comprising an analyte comprising the steps of: (i) eluting the analyte from an ion exchange chromatography column with an ionic eluent to provide an eluate comprising the analyte, wherein the ionic eluent comprises a volatile ionic salt; and (ii) analysing the eluate by mass spectrometry.

3. An apparatus for analysing a sample comprising an analyte, comprising: (i) a reservoir containing an ionic eluent comprising a volatile ionic salt; (ii) an ion exchange chromatography column for eluting the analyte, wherein the column is arranged to receive eluent from the reservoir; and (iii) a mass spectrometer arranged to receive eluate from the column.

4. A method of eluting an analyte from an ion exchange chromatography column, wherein the analyte is eluted using an ionic eluent comprising a volatile ionic salt.

5. (canceled)

6. A saccharide in an ionic buffer, wherein the ionic buffer comprises a volatile ionic salt.

7. The method of claim 2, wherein the sample is a vaccine.

8. The method of claim 2, wherein the analyte is a vaccine.

9. The method of claim 2, wherein the volatile ionic salt is an ammonium salt.

10. The method of claim 9, wherein the volatile ionic salt is ammonium acetate, ammonium benzoate, ammonium bicarbonate, ammonium bromide, ammonium carbamate, ammonium carbonate, ammonium chloride, ammonium formate, ammonium hydrogen phosphate, ammonium hydrogen sulfate, ammonium hydroxide, ammonium nitrate, ammonium oxalate, ammonium phosphate, ammonium sulfate, or ammonium tartrate, or a mixture thereof.

11. The method of claim 10, wherein the volatile ionic salt is ammonium acetate, ammonium bicarbonate, ammonium carbamate, ammonium carbonate, ammonium formate, or ammonium hydroxide, or a mixture thereof.

12. The method of claim 11, wherein the volatile ionic salt is ammonium hydroxide, or ammonium acetate, or a mixture thereof.

13. The eluate obtained by the method of claim 4.

14. A method of analysing the eluate of claim 13 by mass spectrometry.

15. (canceled)

16. The apparatus of claim 3, wherein the sample is a vaccine.

17. The method of claim 4, wherein the analyte is a vaccine.

18. The apparatus of claim 3, wherein the analyte is a vaccine.

19. The method of claim 4, wherein the volatile ionic salt is an ammonium salt.

20. The apparatus of claim 3, wherein the volatile ionic salt is an ammonium salt.

21. The apparatus of claim 20, wherein the volatile ionic salt is ammonium acetate, ammonium benzoate, ammonium bicarbonate, ammonium bromide, ammonium carbamate, ammonium carbonate, ammonium chloride, ammonium formate, ammonium hydrogen phosphate, ammonium hydrogen sulfate, ammonium hydroxide, ammonium nitrate, ammonium oxalate, ammonium phosphate, ammonium sulfate, or ammonium tartrate, or a mixture thereof.

22. The apparatus of claim 21, wherein the volatile ionic salt is ammonium acetate, ammonium bicarbonate, ammonium carbamate, ammonium carbonate, ammonium formate, or ammonium hydroxide, or a mixture thereof.

23. The apparatus of claim 22, wherein the volatile ionic salt is ammonium hydroxide, or ammonium acetate, or a mixture thereof.
